(19) United States
(12) Patent Application Publication
(10) Pub. No.: US 2005/0288532 A1
(43) Pub. Date: Dec. 29, 2005

(54) Oxidation method

(75) Inventors: Thomas Genger, Ludwigshafen (DE); Carsten Oost, Bad Durkheim (DE); Joost-Willem Snoeck, Neustadt (DE); Manfred Stroezel, Ilvesheim (DE); Jens Becker, Eisenberg (DE); Wilfried Berning, Worms (DE)

Correspondence Address: CONNOLLY BOVE LODGE & HUTZ, LLP, P O BOX 2207, WILMINGTON, DE, 19899, US

(73) Assignee: BASF AKTIENGESELLSCHAFT, Ludwigshafen (DE)

(21) Appl. No.: 10/525,468

(22) PCT Filed: Jul. 30, 2003
(86) PCT No.: PCT/EP03/08407

§ 371 Date: Feb. 24, 2005

(30) Foreign Application Priority Data
Aug. 30, 2002 (DE) 102 40 816.5

(51) Int. Cl. C07C 45/27

(52) U.S. Cl. 568/357

(57) ABSTRACT

A process for oxidizing a starting material with an oxidizing agent to obtain a product, which comprises carrying out the oxidation in a reaction apparatus which has a bottom region at the lower end, a top region at the upper end and a reaction zone between the top region and the bottom region, maintaining the reaction mixture in the boiling state in the reaction zone, and introducing oxidizing agent into the reaction zone in at least two substreams.

The present invention relates to a process for oxidizing a starting material with an oxidizing agent to obtain a product,
which comprises
carrying out the oxidation in a reaction apparatus which has
a bottom region at the lower end,
a top region at the upper end and
a reaction zone between the top region and the bottom region,
maintaining the reaction mixture in the boiling state in the reaction zone, and
introducing oxidizing agent into the reaction zone in at least two substreams.

Numerous processes for oxidizing a starting material, in particular an organic starting material, with a molecular oxygen-containing gas to obtain a product are known.

For example, saturated compounds may be converted to unsaturated compounds, such as methylcyclohexane to toluene or propane to propene, alcohols to aldehydes or ketones, such as isopropanol to acetone, s-butanol to methyl ethyl ketone or methanol to formaldehyde, hydrocarbons to hydroperoxides, such as cumene to cumene hydroperoxide, tetralin to tetralin hydroperoxide or cyclohexane to cyclohexane hydroperoxide, olefins to epoxides, such as ethene to ethylene oxide, or hydrocarbons to alcohols, aldehydes, ketones or carboxylic acids, such as cyclohexane to cyclohexanol or cyclohexanone, toluene to benzaldehyde or benzoic acid, o-, m- or p-xylene to the corresponding aromatic dicarboxylic acids or their anhydrides, butane to maleic anhydride or propene to acrolein or acrylic acid.

One of the problems of such oxidation reactions is that the desired products of value may themselves likewise be oxidized to obtain undesired by-products or ultimately carbon dioxide and water. This disadvantageously leads to a reduction in the selectivity of the oxidation reaction.

An industrially significant oxidation is described in: Weissermel/Arpe, Industrielle Organische Chemie, 4th edition, VCH, Weinheim, 1994, pages 260 ff and is the oxidation of cyclohexane to a mixture comprising cyclohexanol and cyclohexanone in the liquid phase using air in the presence of manganese or cobalt salts as catalyst at 125-165° C. and a pressure in the range from 8 to 15 bar (absolute).

In this oxidation, the cyclohexane conversion is limited in order to achieve an industrially viable selectivity. According to Arpentier et al., The Technology of Catalytic Oxidations, Editions Technip 2001, pages 226 ff, the selectivity in cyclohexane conversions in the range of 1-2% is approx. 90%, while even at conversions of 4-5% it falls to 77-85%.

The unconverted cyclohexane has to be distilled off in a downstream distillation column and recycled into the oxidation stage.

Cyclohexanol and cyclohexanone are starting materials for preparing caprolactam and adipic acid which are both in turn used to a considerable extent as monomers for preparing industrially significant polyamides.

DE 19811517 describes the uncatalyzed, selective oxidation of cyclohexane with ozone to cyclohexanone in a reactor inertized toward ozone by metering the ozone in via the top of the column, while at the same time continuously removing the cyclohexanone formed at the bottom of the column as the product.

A disadvantage of this process is the insufficient contact of the oxidizing agent with the starting material and the poor utilization of the oxidizing agent: at industrially relevant pressures, ozone is gaseous and therefore leaves the reactor again without sufficient contact with the hydrocarbon to be oxidized.

Furthermore, the process is intended to be carried out at temperatures less than or equal to the boiling temperature of the cyclohexane to be oxidized. However, since the reaction products boil approx. 75° C. higher than the starting material and the boiling temperature of the reaction mixture is therefore above the boiling temperature of the cyclohexane, this process is a pure liquid phase reaction without distillation. This process therefore has the disadvantages already mentioned above with regard to the separation of the reaction mixture and recycling of the cyclohexane.

It is an object of the present invention to provide a process which facilitates the oxidation of a starting material, in particular an organic starting material, with an oxidizing agent to obtain a product in a technically simple and economical manner while avoiding the disadvantages mentioned.

We have found that this object is achieved by the process defined at the outset.

According to the invention, the present process is suitable for oxidizing a starting material.

Useful starting materials are inorganic, but preferably organic, compounds.

Useful organic compounds may be unsaturated, but preferably saturated, hydrocarbons. In these hydrocarbons, one or more carbon atoms may be replaced by heteroatoms, such as oxygen, nitrogen, sulfur or phosphorus, with the saturation of any free valencies of such heteroatoms by hydrogen or substituents, in particular the substituents specified hereinbelow for the hydrocarbons; preference is given to no carbon atoms being replaced by such heteroatoms. For the purposes of the present invention, the hydrocarbons both with and without such heteroatoms are referred to in summary as hydrocarbons.

Useful unsaturated hydrocarbons include those having one or more triple bonds, one or more olefinic double bonds or aromatic systems, or those which have a combination of such features, such as ethene, propene, 1-butene, 2-butene, 1,3-butadiene, benzene, toluene, o-xylene, m-xylene, p-xylene, fluorene, 2-methylpyridine, 3-methylpyridine, 4-methylpyridine, and tetralin. Useful unsaturated hydrocarbons may be linear or cyclic.

Useful saturated hydrocarbons may be linear or preferably cyclic alkanes, in particular those having from 2 to 12 carbon atoms.

Advantageous linear alkanes are ethane, propane, n-butane, i-butane, n-pentane, n-hexane, n-heptane, n-octane, n-nonane, and n-decane.

Useful cyclic alkanes may be cyclohexane and decalin.

The hydrocarbons may be unsubstituted or substituted, for example by aliphatic groups, preferably C₁-C₈-alkyl groups, such as methyl, ethyl, i-propyl, n-propyl, n-butyl, i-butyl, s-butyl, n-pentyl, n-hexyl, n-heptyl, n-octyl, 2-ethylhexyl, OH, ═O, C₁-C₈-alkoxy, COOH, C₂-C₆-carbalkoxy, C₁-C₁₀-acyloxy or C₁-C₈-alkylamino, sulfonic acid or their salts, such as alkali metal or alkaline earth metal salts, or esters, cyano, or halogens such as fluorine, chlorine or bromine.

In an advantageous embodiment, the process according to the invention may be applied to the oxidation of hydrocarbons or aldehydes to hydroperoxides which may be used, for example, in the indirect epoxidation of olefins, such as acetaldehyde to peracetic acid, isobutane to isobutyl peroxide, isopentane to isopentyl peroxide, ethylbenzene to phenylethyl peroxide, cumene to cumene hydroperoxide, or tetralin to tetralin hydroperoxide.

In a further advantageous embodiment, the process according to the invention may be applied to the oxidation of hydrocarbons or aldehydes to acids or their anhydrides or their ester, such as p-xylene to terephthalic acid, m-xylene to isophthalic acid, o-xylene to phthalic acid or phthalic anhydride, n-butane to acetic acid, toluene to benzaldehyde or benzoic acid, paraffins to acids, acetaldehyde to acetic acid, trimethylbenzene to hemimellitic acid, n-butyraldehyde to n-butyric acid, crotonaldehyde to crotonic acid, butane to ethyl acetate, butene to maleic anhydride, butane to maleic anhydride, benzene to maleic anhydride, or propene to acrylic acid.

In a further advantageous embodiment, the process according to the invention may be applied to the oxidation of hydrocarbons or aldehydes to ketones, alcohols or quinones, such as fluorene to fluorenone, trimethylphenol to trimethylquinone, acetaldehyde to acetic anhydride, naphthalene to naphthoquinone, anthracene to anthraquinone, p-diisopropylbenzene to hydroquinone, p-methylisopropylbenzene to cresol, or paraffins to alcohols. In a further advantageous embodiment, the process according to the invention may be applied to the oxidation of alcohols to aldehydes or ketones, such as isopropanol to acetone, s-butanol to methyl ethyl ketone, or methanol to formaldehyde.

In a further advantageous embodiment, the process according to the invention may be applied to the oxidation of C—C single bonds to C—C multiple bonds, such as butene to butadiene, ethylbenzene to styrene, methylcyclohexane to toluene, or propane to propene.

In a further advantageous embodiment, the process according to the invention may be applied to the oxidation of hydrocarbons to nitriles, such as the oxidation of toluene with N₂O to benzonitrile.

In a further preferred embodiment, the process according to the invention may be applied to the oxidation of C—C single bonds or C—C multiple bonds using ozone to obtain an acid function, such as the ozonolysis of natural products to fatty acids.

In a further advantageous embodiment, the process according to the invention may be applied to the oxidation of C—C multiple bonds using hydrogen peroxide to obtain the corresponding diols, such as allyl alcohol to glycerol.

The hydrocarbons may be used as individual compounds or as a mixture of such hydrocarbons.

In a particularly preferred embodiment, the starting material used may be cyclohexane.

Advantageous products in this case are cyclohexanol, cyclohexanone, cyclohexyl hydroperoxide or their mixtures, in particular cyclohexanol, cyclohexanone or their mixtures.

According to the invention, a starting material is oxidized using an oxidizing agent.

In an advantageous embodiment, the oxidizing agent used may be a molecular oxygen-containing gas, in particular molecular oxygen.

The molecular oxygen used may be dioxygen in the triplet or singlet state or trioxygen, i.e. ozone, preferably dioxygen, in particular in the triplet state, or mixtures of such molecular forms of oxygen. The gas comprising such molecular oxygen may be free of further components.

The gas containing such molecular oxygen may comprise further, different components.

Useful further, different components include oxidizing gases, such as nitrogen oxides.

In the case of further, different components, it may be preferable to use inert gases, i.e. those which do not enter substantially into the oxidation reaction, if at all, in the process according to the invention, such as nitrogen, for example in the form of air, or noble gases, for example, argon, or their mixtures.

In a further preferred embodiment, the oxidizing agent used may be a gas comprising one or more nitrogen oxides, in particular one or more nitrogen oxides.

Useful nitrogen oxides include dinitrogen monoxide, nitrogen monoxide, nitrogen dioxide, and their mixtures or oligomers. The gas comprising one or more such nitrogen oxides may be free of further components.

The gas comprising one or more such nitrogen oxides may contain further, different components.

Useful further, different components include oxidizing gases, such as oxygen.

In the case of further, different components, it is advantageous to use inert gases, i.e. those which do not enter substantially into the oxidation reaction, if at all, in the process according to the invention, such as nitrogen, for example in the form of air, or noble gases, for example argon, or their mixtures.

In a further preferred embodiment, the oxidizing agent used may be a compound which is liquid under the reaction conditions, such as a peroxide, for example an inorganic peroxide, such as hydrogen peroxide, or an organic peroxide, such as cyclohexane hydroperoxide, isobutyl hydroperoxide, isopentyl hydroperoxide, phenylethyl hydroperoxide, cumene hydroperoxide, tetralin hydroperoxide, or a peracid, such as peracetic acid.

The mixing ratios between starting material used and the molecular oxygen in the molecular oxygen-containing gas depends on the desired degree of conversion of the starting material to the product from a chemical point of view, for example the conversion of an alkane to an alcohol or a ketone, and from a process engineering point of view, i.e. the desired conversion, and may be easily optimized by a few simple preliminary experiments.

Oxidizing agent and starting material may be added separately to the reaction apparatus.

Oxidizing agent and starting material can be partially mixed before addition to the reaction apparatus and added to the reaction apparatus.

Oxidizing agent and starting material can be completely mixed before addition to the reaction apparatus and added to the reaction apparatus.

According to the invention, the oxidation is carried out in a reaction apparatus which has
a bottom region at the lower end,
a top region at the upper end and
a reaction zone between the top region and the bottom region.

Preferred reaction apparatus are rectification columns, as described, for example, in Kirk-Othmer, Encyclopedia of Chemical Technology, 3. Ed., Vol. 7, John Wiley & Sons, New York, 1979, pages 870-881, such as tray columns, for example sieve tray columns or bubble-cap tray columns, or columns having structured packings or random packings.

In a preferred embodiment, useful trays are those which facilitate a long residence time of the reaction mixture in the column, such as valve trays, preferably bubble-cap trays or tunnel-cap trays.

In a further preferred embodiment, structured packings, such as woven metal packings or sheet metal packings, advantageously having an ordered structure, or random packings are contemplated.

In a further preferred embodiment, hold-up packings are considered. Such hold-up packings allow the residence time in the reaction zone to be adjusted with the aid of the pressure drop and, even at high load, ensure a good separation performance.

In a particularly preferred embodiment, it is possible to use internals having a high number of plates, such as woven metal packings or sheet metal packings, advantageously having an ordered structure, below the lowermost feedpoint for the oxidizing agent into the reaction apparatus.

Advantageously, the rectification column should have a separation performance of from 10 to 100, preferably from 20 to 40, theoretical plates.

Advantageously, the higher-boiling reactant of the two reactants starting material and oxidizing agent may be fed to the reaction apparatus predominantly or completely above the lower-boiling reactant, and in particular, the higher-boiling reactant may be fed into the upper section of the rectification column and the lower-boiling reactant into the lower section of the rectification column.

The higher-boiling reactant may comprise lower-boiling reactant.

The lower-boiling reactant may comprise higher-boiling reactant.

In a particularly preferred embodiment, the rectification column has a distillation section between the reaction section and bottom.

It has proven particularly advantageous to install from 0 to 50, preferably from 5 to 30, theoretical plates in the lower section of the rectification column, i.e. the distillation section.

It has proven particularly advantageous to install from 0 to 50, preferably from 5 to 30, theoretical plates in the upper section of the rectification column, i.e. the reaction zone. The reaction zone may be situated within the rectification section of the column.

The reaction zone may be situated outside the rectification section of the column.

The reaction zone may be situated outside the rectification column.

In this case, the pressure in the reaction zone and the pressure in the rectification column may be the same or different.

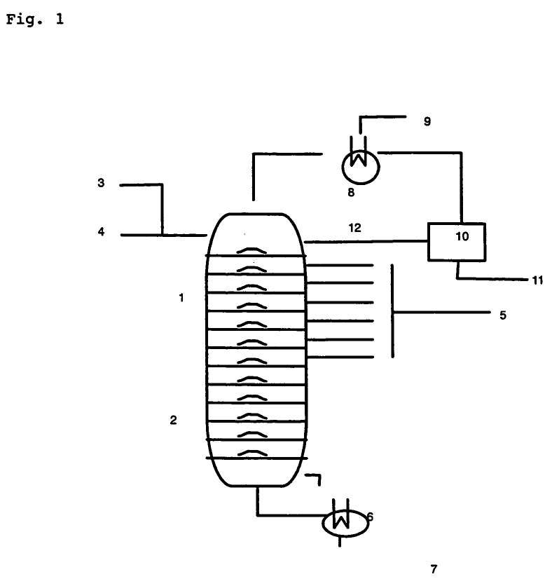

FIG. 1 shows a schematic of an advantageous embodiment of a reaction apparatus. In FIG. 1:

1: reaction section

2: distillation section

3: feed for starting material

4: feed for catalyst

5: addition of oxidizing agent, in particular gaseous oxidizing agent, such as air

6: evaporator

7: product stream

8: heat exchanger

9: discharge of inerts

10: separator

11: water discharge

12: starting material recycling

The process according to the invention may preferably be carried out in a plurality of reaction apparatus connected in series. When operating the downstream reaction apparatus at a lower pressure, a portion of the energy contained in the vapor stream of the upstream column may advantageously be transferred to the feed stream of one of the downstream reaction apparatus.

Furthermore, a portion of the uncondensed vapor stream may advantageously be recycled into the lower section of the reaction apparatus. This cycle gas method allows a portion of the energy present in the bottom stream to be recovered.

The average residence time of the reaction mixture on the trays of the column should be from 1 to 120 minutes, preferably from 5 to 30 minutes.

The process according to the invention, in particular when cyclohexane is used as the starting material, may preferably be carried out at a pressure in the range from 0.1 to 3.5 MPa, preferably from 0.5 to 2.5 MPa, measured in the bottom region of the reaction apparatus.

The temperature is then considered with regard to the maintenance in the boiling state of the reaction mixture in the reaction zone. The temperature suitable for this purpose for the particular reaction may be easily determined by a few simple preliminary experiments.

When cyclohexane is used as the starting material, advantageous temperatures in the reaction zone are in the range from 70 to 220° C., preferably from 120 to 190° C.

In a further preferred embodiment, the reaction apparatus may have a means for withdrawing gases at the upper end of the upper section.

Advantageously, the reaction is carried out in such a way that reaction mixture present below the reaction zone is evaporated to obtain a mixture of liquid and gaseous reaction mixture.

In an advantageous embodiment, the reaction apparatus is filled with liquid reaction mixture in the bottom region and in the region of the reaction zone.

Owing to its lower density compared to the liquid reaction mixture, the gaseous reaction mixture obtained in this way then rises in the direction of the top region of the reaction apparatus. Owing to the interaction between the gaseous and the liquid phase, condensation and evaporation processes may result in changes in the composition of the gas phase.

According to the invention, the gaseous reaction mixture arriving in the top region of the reaction apparatus is condensed and fed thus to the reaction zone, advantageously in the liquid phase.

According to the invention, the oxidizing agent is introduced into the reaction zone in at least 2, preferably from 2 to 100, in particular from 2 to 50, more preferably from 2 to 40, such as 2, 3, 4, 5, 6, 7, 8, 9, 10, 11, 12, 13, 14, 15, 16, 17, 18, 19, 20, substreams.

The oxidizing agent may be introduced into the reaction apparatus by processes known per se, in particular for the introduction of a gas into a liquid.

The process according to the invention may be carried out without a catalyst.

The process according to the invention may be carried out in the presence of a homogeneous or heterogeneous catalyst.

When a homogeneous catalyst is used, this may advantageously be added to the reaction mixture in the top region of the reaction apparatus and withdrawn with the reaction mixture in the bottom region.

When a heterogeneous catalyst is used, this may advantageously be immobilized in the reaction zone of the reaction apparatus by processes known per se.

In general, catalysts known per se may be used for the particular oxidation reactions, for example, in the case of the oxidation of cyclohexane to cyclohexanol, cyclohexanone or its mixtures, cobalt or manganese salts.

The amounts of catalyst may easily be determined in accordance with the catalyst velocities known for these catalysts for the particular reactions and the conversions selected in the process according to the invention, and an optimization of the catalyst amounts may easily be carried out by a few simple preliminary experiments.

Advantageously, a reaction mixture comprising the product may be withdrawn in the bottom region of the reaction apparatus, in particular when the boiling point of the product is higher than the boiling point of the starting material under the reaction conditions. The reaction mixture withdrawn in the bottom region may consist of product or a mixture which comprises the product in addition to further components, such as starting material, by-products and secondary products.

Advantageously, a reaction mixture comprising the product may be withdrawn in the top region of the reaction apparatus, in particular when the boiling point of the product is lower than the boiling point of the starting material under the reaction conditions. The reaction mixture withdrawn in the top region may consist of product or a mixture which comprises the product in addition to further components, such as starting material, by-products and secondary products.

When water is to occur in the oxidation reaction according to the invention as an inevitable or undesired by-product or as a secondary product, this may advantageously be withdrawn during the oxidation from the reaction apparatus above the reaction zone, advantageously overhead.

### COMPARATIVE EXAMPLE 1

In a bubble column reactor divided into eight chambers, the cyclohexane stream which had been added at the upper end of the reactor was adjusted in such a way that the residence time of the liquid phase in the reactor was 31 minutes. By adding an appropriate amount of air evenly distributed over the chambers of the reactor, a cyclohexane conversion of 3.5% was set. The reactor was operated at a pressure of 16 bar.

The total selectivity for cyclohexanol, cyclohexanone and cyclohexane hydroperoxide was 83.9%. The space-time yield, based on the liquid phase in the reactor, was 45.7 kg/(m³*h).

### EXAMPLE 1

2415 kg/(m³*h) of cyclohexane, based on the liquid phase volume, were fed above the reaction section to a reaction column having 10 trays in the reaction section (upper) and 10 trays in the distillation section (lower). The column was operated at a pressure of 11.9 bar. 0.15 m³ (STP) of air per kg of cyclohexane was introduced uniformly distributed over the 10 trays of the reaction section of the column. At an evaporator energy based on the fresh cyclohexane stream of 200 Wh/kg, the cyclohexane conversion was 10.1%.

The total selectivity for cyclohexanol, cyclohexanone and cyclohexane hydroperoxide was 88.0%. The space-time yield based on the liquid phase in the reactor was 250 kg/(m³*h).

### COMPARATIVE EXAMPLE 2

Example 1 was repeated, with the difference that all of the air was introduced in one stream into the lowermost tray of the reaction section.

The cyclohexane conversion was 9.8%.

The total selectivity for cyclohexanol, cyclohexanone and cyclohexane hydroperoxide was 84.1%. The space-time yield, based on the liquid phase in the reactor, was 232 kg/(m³*h).

1. A process for oxidizing a starting material with an oxidizing agent to obtain a product
which comprises
carrying out the oxidation in a reaction apparatus which has
a bottom region at the lower end,
a top region at the upper end and
a reaction zone between the top region and the bottom region,
maintaining the reaction mixture in the boiling state in the reaction zone,
and
introducing oxidizing agent into the reaction zone in at least two substreams.

2. A process as claimed in claim 1, wherein the unconverted starting material leaving the reaction zone is recycled into the reaction zone.

3. A process as claimed in claim 1, wherein the starting material used is a linear or cyclic alkane.

4. A process as claimed in claim 1, wherein the oxidizing agent used is an oxidizing agent which is gaseous under the reaction conditions.

5. A process as claimed in claim 4, wherein the oxidizing agent used is a molecular oxygen-containing gas.

6. A process as claimed in claim 1, wherein the oxidation is carried out in the presence of a catalyst.

7. A process as claimed in claim 1, wherein water is by-produced in the oxidation and this water is withdrawn during the oxidation from the reaction apparatus in the reaction zone or in the top region.

8. A process as claimed in claim 1, which is carried out at a temperature in the range from 10 to 300° C., measured in the reaction zone.

9. A process as claimed in claim 1, wherein the reaction apparatus used is a rectification column.

10. A process as claimed in claim 1, wherein the starting material is oxidized with cycle gas which is enriched with an oxidizing agent.

11. A process as claimed in claim 1, wherein a product-containing reaction mixture is withdrawn below the reaction zone.

12. A process as claimed in claim 1, wherein the higher-boiling reactant selected from the group consisting of oxidizing agent and starting material is fed to the reaction apparatus above the lower-boiling reactant selected from the group consisting of oxidizing agent and starting material.

13. A process as claimed in claim 1, wherein the starting material used is cyclohexane.

14. A process as claimed in claim 1, wherein cyclohexane is oxidized with air, reaction mixture is continuously withdrawn in the bottom region of the reaction apparatus and unconverted cyclohexane and water are continuously removed in the top region, cyclohexane and water are separated by means of a phase separator and the resulting cyclohexane is fed to the top region of the reaction apparatus as reflux.

15. A process as claimed in claim 2, wherein the starting material used is a linear or cyclic alkane and the oxidizing agent used is an oxidizing agent which is gaseous under the reaction conditions.

16. A process as claimed in claim 15, wherein the oxidation is carried out in the presence of a catalyst and water is by-produced in the oxidation and this water is withdrawn during the oxidation from the reaction apparatus in the reaction zone or in the top region.

17. A process as claimed in claim 16, which is carried out at a temperature in the range from 10 to 300° C., measured in the reaction zone and the reaction apparatus used is a rectification column.
